(19) United States
(12) Patent Application Publication
(10) Pub. No.: US 2010/0256581 A1
(43) Pub. Date: Oct. 7, 2010

(54) Prefilter for an Ostomy Bag

(75) Inventor: Lasse Skoett Albrectsen, Frederiksberg (DK)

Correspondence Address: COLOPLAST A/S;Attention: Corporate Patents, Holtedam 1, DK-3050 Humlebaek, DK

(73) Assignee: COLOPLAST A/S, Humlebaek (DK)

(21) Appl. No.: 12/746,176

(22) PCT Filed: Dec. 16, 2008
(86) PCT No.: PCT/DK08/50314

§ 371 Date: Jun. 4, 2010

(30) Foreign Application Priority Data
Dec. 20, 2007 (DK) PA 2007 01829

(51) Int. Cl. A61F 5/441

(52) U.S. Cl. 604/333

(57) ABSTRACT

An ostomy appliance of the kind comprising a collecting bag for waste material, at least one opening through which waste material may enter the collecting bag, at least one filter allowing gas to exit the collecting bag and at least one pressure sensitive gas permeable barrier-filter positioned between the at least one opening and the at least one filter, where the barrier-filter in its closed state blocks the passage of semi-solid waste and in its open state maintains an open passage for semi solid waste, where an increase in pressure inside the collecting bag passes a predefined first gas pressure limit triggering the transition of the barrier-filter from its closed state to its open state.

### FIELD OF THE INVENTION

The present invention relates to ostomy appliances having a collecting bag, where the collecting bag has an opening for collecting waste material, a deodorizing gas filter and a pressure sensitive gas permeable barrier-filter positioned between the opening and the deodorizing gas filter, where the barrier-filter protects the deodorizing filter from being clogged by semi-solid waste when closed.

### BACKGROUND

One problem with the use of ostomy appliances is that the ostomy appliance collects not only the solid and/or liquid waste material from the intestine of the user, but also the discharged flatus gas. The volume of the flatus may be significantly larger than the volume of the other waste material, and this might cause the ostomy appliance to expand and balloon, as the flatus cannot escape from the ostomy appliance. The ballooning may cause the user considerable pain, if the ostomy appliance expands to its maximum capability as the flatus gas can only escape the intestine via the stoma and the ostomy appliance. The ballooning may further be problematic as it may cause unwanted deformation of the adhesive connecting means used to secure the ostomy appliance to the user. The unwanted deformation may partly release the connecting means from the skin surface of the user and open a passage causing leakage of solid and/or liquid waste material from the ostomy appliance, which would be uncomfortable and/or embarrassing for the user.

A solution to this problem has been presented in WO 2004/004612, which describes a colostomy pouch having a deodorizing gas release filter, which filters the flatus gas before the gas is released from the colostomy pouch to the outside environment.

A major problem concerning the use of gas release filters in ostomy appliances is that when solid and/or liquid waste material is collected into an appliance there is a considerable risk that the gas release filter becomes clogged or blocked by the waste material. One proposed solution to this problem is presented in WO 2004/004612, where the front and back panels of the colostomy pouch have been secured together along a line extending between the filter and the aperture of the pouch, where the line has at least one gap where the gas may escape from the pouch towards the filter restricting the faecal matter from flowing towards the filter.

The above-mentioned solution might be problematic as faecal matter may clog and/or block the at least one gap, blocking the pathway, and the enclosed gas cannot escape the colostomy pouch via the filter. Thus, the pouch has to be removed from the user and the gas would be released via the aperture of the pouch.

Therefore, there is a need for an ostomy appliance, preferably an ostomy pouch, which has a gas permeable barrier separating the area holding the waste material and the gas release filter, which may be unclogged or unblocked without having to remove the ostomy pouch from the user.

### SUMMARY

An object of the present invention is to provide an ostomy appliance of the kind that may automatically open a clogged or a blocked barrier-filter, allowing unwanted flatus gas to escape the ostomy appliance.

Another object of the present invention is to provide an ostomy appliance of the kind that allows the user to manually open a clogged or a blocked barrier-filter, allowing unwanted flatus gas to escape the ostomy appliance.

A further object of the present invention is to provide an ostomy appliance having a barrier-filter that can be closed after the release of unwanted flatus gas, again providing a barrier for semi-solid waste material between the opening and the filter.

### DETAILED DESCRIPTION OF THE INVENTION

The present invention relates to an ostomy appliance of the kind comprising a collecting bag for waste material, at least one opening through which waste material may enter the collecting bag, at least one filter allowing gas to exit the collecting bag, and at least one pressure sensitive gas permeable barrier-filter positioned between the at least one opening and the at least one filter, where the barrier-filter, in its closed state, blocks the passage of semi-solid waste and, in its open state, maintains an open passage for semi-solid waste, where an increase in pressure inside the collecting bag exceeding a predefined first gas pressure limit triggers the transition of the barrier-filter from its closed state to its open state.

This means that any unwanted flatus gas collected in the ostomy appliance may easily be removed from the bag without the removal of the ostomy appliance from the user. In its closed state, the barrier-filter hinders the semi-solid waste material from reaching the filter, but as the barrier-filter is gas permeable the flatus gas is free to communicate from one side of the barrier-filter to the other. The primary cause for clogged filters in ostomy appliances is the direct contact to collected semi-solid waste. By providing a barrier-filter, which separates the opening through which the waste enters and the filter, the risk of filter blockage is significantly reduced, as the waste material is not in direct communication with the filter when the barrier-filter is in its closed state.

When the barrier-filter is in its closed state, there is a considerable risk that any collected semi-solid waste may clog and/or block the barrier-filter, blocking the gas communication across the filter, and any collected flatus gas cannot escape the ostomy appliance. Thus, to enable the escape of the collected flatus gas from the ostomy appliance, the barrier-filter must be unclogged and/or unblocked resuming the gas communication across the barrier filter.

If the barrier-filter is blocked and flatus gas continuously enters the ostomy appliance from the stoma via the opening through which waste enters, the pressure inside the ostomy appliance increases as the gas cannot escape the appliance. The increased pressure might cause the appliance to balloon, increasing the risk of leakage and possibly causing embarrassment and/or discomfort to the user. In the present invention, the barrier filter is pressure sensitive, where an increased pressure within the ostomy appliance causes a physical and/or chemical reaction within the barrier-filter when a first gas pressure limit is passed. This has the result that the barrier-filter opens a passage providing a direct communication pathway between the collection bag holding the semi-solid waste and flatus gas and the filter.

Within the context of the present invention, the term barrier-filter means a preceding filter or a pre-filter which provides a certain degree of filtering to a substance, element, composition, mixtures of different substances or matter, if the aforementioned is subsequently filtered using a main, a primary or a principal filter. The function of the pre-filter is to filter out elements which may be harmful or disadvantageous when in contact with the main or the principal filter.

Within the context of the present invention, the term pressure is defined as the pressure increase with regards to the pressure environment that the ostomy appliance is situated in. This means that when a pressure value is mentioned, e.g. 1 kPa, the actual pressure measurement would be the surrounding pressure (for example atmospheric pressure=1.013*10² kPa)+1 kPa.

The ostomy appliance according to the present invention may be a one-piece ostomy appliance having a permanent adhesive wafer for connecting the opening to a body orifice, i.e. a stoma, or it might be a two-piece ostomy appliance where a coupling element is in temporary connection with the collection bag. The two-piece appliance has an adhesive wafer fastened to the user around the stoma and the bag may be coupled to and removed from the adhesive wafer at will. The use of a one-piece and a two-piece ostomy appliance is well known within the art.

In one embodiment of the present invention the barrier-filter may be of the resealable kind. This means that if a barrier-filter has been through a transition from a closed state to an open state, the barrier-filter is suited to return from the open state to the closed state at least once. A resealable barrier-filter provides a barrier-filter which is able to go through the transition from a closed state to an open state multiple times. Such a barrier-filter can shift from its open state to its closed state every single time, assuming there is no physical hindrance preventing the barrier-filter from resealing to its closed state.

In the context of this application, a physical hindrance which prevents the barrier-filter from resealing is when the ostomy appliance is filled with semi-solid waste material up to a level where the waste material cannot be maneuvered or moved away from the open barrier-filter. Another physical hindrance is if the barrier-filter is contaminated with semi-solid waste material such that the means providing the physical and/or chemical reaction are incapacitated and the barrier filter is incapable of reacting to physical changes within the ostomy appliance.

In one embodiment of the present invention, the barrier-filter may return from its open state to its closed state when the pressure inside the collecting bag is decreased below a predefined second gas pressure limit. This means that if the barrier-filter has been triggered to its open state or if the barrier-filter is in its open state and the pressure level inside the ostomy appliance decreases to a level below a predefined second gas pressure limit, the barrier-filter returns to its closed state. Furthermore, if the barrier-filter has accidentally or inadvertently been triggered to its open state and the pressure level is below the second gas pressure limit, the barrier-filter returns to its closed state.

During use, the ostomy appliance usually collects the flatus gas secreted from the intestine of the user. In a situation where the barrier-filter is blocked or clogged and the user notices that the collected flatus gas cannot escape the ostomy appliance, the user can actively stimulate the ostomy appliance, triggering the transition of the barrier-filter from its closed state to its open state. In one embodiment of the present invention, the pressure inside the collecting bag may be increased by applying pressure to the external surface of the collecting bag. This means that the user may use his extremities to apply pressure on the surface area of the ostomy appliance, increasing the internal pressure and as the pressure passes the first gas pressure limit, the barrier-filter goes from the closed state to the open state, opening a communication pathway from the collecting bag to the filter. For example, in a situation where the ostomy appliance is hidden underneath the clothes of a user and the appliance begins to balloon, the user can increase the internal pressure manually, allowing the flatus gas to escape, thereby deflating the appliance before the appliance can cause the user discomfort or embarrassment.

In one embodiment of the present invention the barrier-filter may be a hook and loop fastener filter and in a related embodiment the barrier-filter may be a Velcro filter. The Velcro filter comprises a hook part and a loop part, where each individual part has an inner surface and an outer surface. The inner surface of each individual part comprises the hook or loop fasteners, respectively, and the outer surface may be permanently connected to a suitable surface area. The outer surfaces of the hook part and the loop part may be secured to the inner surface of the ostomy appliance, for example on the inner surface of the front-wall and back-wall, respectively, where the inner surfaces of the hook part and the loop parts are positioned opposite each other. When the inner surface of the hook part and the loop part of the Velcro filter is in contact with each other, the Velcro filter provides a barrier which is impermeable to semi-solid material, such as collected faeces, and is permeable to gas. Thus, in this state, the Velcro filter barrier-filter prevents the semi-solid material from reaching the deodorizing filter and reduces the risk of filter clogging or blockage significantly.

When the hook-part and the loop-part of the Velcro filter are in contact with each other, a certain amount of force is needed to separate the parts from each other. The specific amount of force needed is highly dependent on what type of Velcro is used. Some types of Velcro require a relatively low separation force while other types require more force. There are a few important factors when assessing the force required to separate the hooks and the loops such as the number of hooks and loops per area unit, the angle of the force applied for separating the parts from each other, and the strength of the hook part and the loop part.

In order to provide an ostomy appliance having a Velcro filter which operates as a barrier-filter which may be transformed from a closed state to an open state and vice versa using a first and a second gas pressure limit, there are several factors which have to be considered when choosing the gas pressure limits and construction of the ostomy appliance. Such factors may be the positioning of the barrier-filter inside the ostomy appliance, the shape and size of the barrier-filter, the choice of barrier-filter material, the shape and size of the front and back walls of the ostomy appliance and the choice of material of the walls. The influences of the different factors are obvious to the skilled person based on the teachings in the present application.

The separation force, barrier and flow properties of the Velcro can be adjusted to optimal performance by choosing the right type and density of hook and loop and physical shape of the Velcro. The barrier-filter properties ensure that only gas passes through the Velcro. When semi-solid waste (of any viscosity) or stool reaches the Velcro barrier, the fine hook and loop fibres hinder the stool in penetrating the Velcro barrier. If pressure builds up inside the bag, the Velcro barrier-filter is designed to separate before the stool penetrates the Velcro barrier. The Velcro separation force can be optimised to separate at an exactly chosen force by the physical design of the Velcro barrier-filter, choice of hook and loop and the positioning of the barrier-filter. A large barrier filter, which has a larger contact surface of hook and loop connections, requires more force to open than a barrier filter having the same type of Velcro, but having a smaller contact surface. The size of the contact surface may be adjusted evenly across the barrier-filter or the size of the surface may be adjusted in an arbitrary manner, provided that the barrier-filter provides an effective barrier for semi-solid waste.

The gas permeable Velcro barrier-filter will, when blocked or contaminated with stool, separate and allow gas to access the deodorizing filter directly. The Velcro barrier can be succeeded by one or more second Velcro barrier-filters or by one or more barriers having different properties like a super absorbent material. By combining one or more Velcro barriers with other barriers a durable filter solution can be obtained.

In one embodiment of the present invention the barrier-filter may be constructed using a material which has similar mechanical properties to the hook and loop. One example of such a material might be the Aplix® 200 or Aplix® 220, which are mushroom fastening systems where mushroom heads interlock, and the Aplix® 100 Knit Loops. It would be obvious for the skilled person, based on the teachings of the present invention, that other types of hook and loop or similar systems might be used.

In accordance with the teachings of the present invention it is obvious to the skilled person that the barrier-filter may be constructed using other types of material. One example could be a woven or a non-woven gas permeable fabric, kept in its closed state using an adhesive surface, which provides a temporary connection with the front or back-wall of the ostomy appliance. The gas may penetrate the fabric, but the fabric blocks semi-solid waste. In an open state, the temporary adhesive connection is disconnected from the front or back wall and a communication pathway is open. Another example might be a gas permeable material having a first part connected to the inside of one wall and a second part connected to the inside surface of the opposing wall and the two parts have opposing adhesive surfaces which may temporarily connect to each other in their closed state and may separate from each other in their open state. A third example might be barrier-filters similar to those in the previous examples, where the adhesive surface or surfaces are replaced with magnetic surfaces providing the temporary connection.

In one embodiment of the present invention the external surface of the front-wall or the back-wall of the ostomy appliance may be provided with a pulling tab, which is permanently fastened to the external surface. The pulling tab could be positioned adjacent to or opposite to the first or the second part of the barrier-filter. The user would pull the tab, thereby applying tensional forces to the front-wall or the back wall providing enough separation force to separate the walls of the barrier filter, which in turn would open a passage through the barrier filter.

In a preferred embodiment of the present invention the collecting bag may be of the kind comprising at least a front-wall and a back-wall, having their respective inner and outer surfaces where the front wall faces away from the user and the back-wall faces the user when connected. The inner surfaces of the front-wall and the back-wall of the collecting bag are in permanent connection to each other at the distal edges of the walls, where the permanent connection is provided by techniques such as laser welding, heat welding, adhesive bonding or similar methods known in the art. This ensures that any waste material collected in the bag cannot escape from the bag through the distal edges of the front-wall and the back-wall.

The opening of the ostomy appliance is positioned on the back-wall such that the waste material secreted by the stoma does not block the subsequent secretion from the stoma. It is well known within the art that the opening of an ostomy appliance is placed in a direction close to the top edge of the ostomy appliance, such that the waste material can be collected in the collection bag in a direction towards the bottom edge of the ostomy appliance. The collected semi-solid waste or stool is of a higher density than the gasses collected in the bag, such that the gasses are generally on top of the semi-solid waste when viewed in the direction of gravity. Thus, the filter may be positioned close to and/or near the top edge of the ostomy appliance, such that the gasses do not have to communicate through the semi-solid waste to escape from the ostomy appliance via the filter.

In one embodiment of the present invention the barrier filter may be secured to the inner surface of the front-wall and the back-wall of the ostomy appliance. The barrier filter is advantageously provided with at least two surface areas that are suited to be permanently connected to the inner surface of the front-wall and the back-wall of the ostomy appliance. This means that the barrier-filter separates an upper volume of the collecting bag, which is close to the top edge of the ostomy appliance, from a lower volume of the collecting bag, which is close to the bottom part of the collecting bag. The barrier-filter stretches from one area of the permanent connection between the front-wall and the back-wall to another area of the permanent connection, such that the barrier filter, in a closed state, prevents any semi-solid waste from reaching the filter located close to the top edge of the ostomy appliance, allowing the gaseous substance to communicate from the lower volume to the upper volume via the barrier-filter.

In one embodiment of the present invention the barrier-filter is laser welded, heat welded and/or adhesively bonded to the inner surface of the front-wall and the back-wall of the ostomy appliance. By using the above-mentioned means for securing the barrier-filter to the ostomy appliance, the barrier-filter is permanently secured to the inner surface of the ostomy appliance. The securing means are preferably applied to the barrier filter such that the permanent connection between the barrier filter and the inner surface of the ostomy appliance is not permeable to any substance which is intended to be collected in an ostomy appliance as known in the art. Thus, any waste substance present in the ostomy appliance preferably has only one way to communicate from the bottom-part to the top part, that is via the barrier-filter as earlier described.

In one embodiment of the present invention the first pressure limit (for triggering the transition from the closed state to the open state) is in the range between 0.1 and 10 kPa, such as between 0.5 and 5 kPa, such as between 0.9 and 1.1 kPa, such as 0.95 and 1.05 kPa. The preferred first pressure limit may vary from one embodiment to the next, where different embodiments of the present inventions may have different first pressure limits. There are different scenarios where different pressure limits may be relevant in various situations that a typical user might be facing. For example, if the user is attending a formal gathering it may be embarrassing if the ostomy appliance begins to balloon. Therefore, a specific ostomy appliance might be fitted with a barrier-filter having a first pressure limit set close to the minimum value of the previously mentioned range, allowing the barrier-filter to open at a minimal increase in internal pressure. However, in situations where the user is in his/her private surroundings, the ballooning of the ostomy appliance might be acceptable. Therefore, the first pressure limit might be set at a higher value, compared to the previous situation, where more internal pressure is allowed before the barrier-filter would open a direct communication pathway from the lower volume to the upper volume.

It may be assumed that the barrier-filter has an upper limit for how many times it can go from its open state to its closed state, and vice versa, which means that an ostomy appliance having a low first gas pressure limit and opens early would have a shorter life span than one having a higher first gas pressure limit, as the first one opens at a higher frequency than the second one. The shorter life cycle may be contributed to wear and tear of the barrier-filter or contamination to the barrier-filter's temporary connection surfaces.

In one embodiment of the present invention, the second pressure limit (for triggering the transition from the open state to the closed state) is in the range between 0 and 0.1 kPa, such as between 0 and 0.05 kPa, such as between 0 and 0.01 kPa. Setting a second pressure limit ensures that when the barrier-filter of the ostomy appliance is in its open state and the pressure within the appliance has gone below the predefined second pressure limit, the barrier filter closes the direct communication pathway between the lower volume and the upper volume of the ostomy appliance, where the barrier-filter protects the filter from contamination of semi-solid waste.

The ostomy appliance filter functions as a gas filter, which removes certain impurities from the gas, passed through the filter. Usually, the removed impurities are offensive odorizing elements, which might cause embarrassment to the user or be uncomfortable if passed unfiltered into the atmosphere. In one embodiment of the present invention, the filter may comprise at least one deodorizing gas filter. The filters allows the user to function in a plurality of different public or social situations, without having to worry about inadvertently discharging unwanted flatus gas having an unpleasant odour from the ostomy appliance.

### BRIEF DESCRIPTION OF THE DRAWINGS

The invention will now be described in further detail below with reference to the schematic drawings in which,

FIG. 1 is an exploded view of an ostomy appliance according to the present invention,

FIG. 2 is a partial planar view of an ostomy appliance according to the present invention,

FIG. 3a is a partial sectional side view of an ostomy appliance along line III-III in FIG. 1 according to the present invention, showing an empty ostomy appliance having the barrier-filter in a closed state,

FIGS. 3b and 3b′ are the same showing a ballooned ostomy appliance having the barrier-filter in a closed state and the forces affecting the barrier-filter, respectively,

FIG. 3c is the same showing a ballooned ostomy appliance having the barrier-filter in an open state, and

FIG. 4 is a plane view of an example of an ostomy appliance according to the present invention.

### DETAILED DESCRIPTION OF THE DRAWINGS

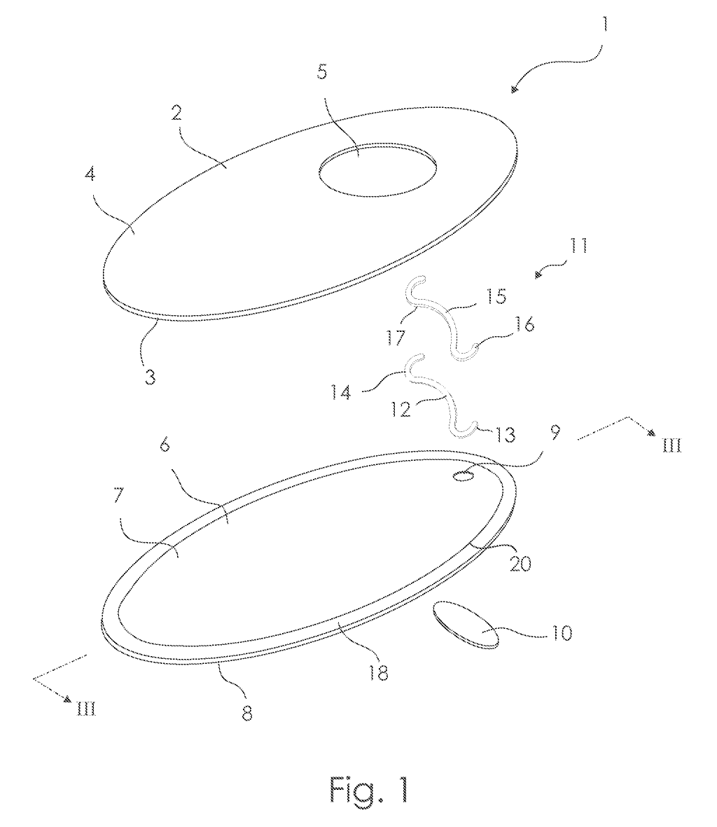

FIG. 1 is an exploded view of an ostomy appliance 1 according to the present invention, having a back-wall 2, which has an internal surface 3 and an external surface 4. The back wall is provided with an opening 5, which functions as the opening where waste material may enter the ostomy appliance 1. The front-wall 6 of the ostomy appliance is substantially of the same circumference size as the back-wall 2 and the front-wall has an internal surface 7 and an external surface 8.

In this embodiment of the present invention the front-wall 6 is provided with a filter opening 9 that is in direct communication with a deodorizing gas filter 10, which allows enclosed gas to escape from the ostomy appliance 1. The deodorizing gas filter 10 is of the kind which is gas permeable and impermeable to the semi-solid waste and ensures that the semi-solid waste cannot escape the ostomy appliance via the filter opening 9. The deodorizing filter 10 is connected to the ostomy appliance 1 using permanent connection means, in this embodiment on the external surface 8 of the front-wall 6 where the surface area of the filter 10 is larger than the area of the filter opening 9, and the filter 10 covers the filter opening 9 completely. An example of a deodorizing filter 10 known in the art which is positioned on the outside of the ostomy appliance may be found in European patent EP0089110. In alternative embodiments of the present invention, the filter 10 may be of the kind which can be placed inside the ostomy appliance 1, where the filter 10 covers the filter opening 9 completely on the inner surface 3,7 of the front 6 or back-wall 2 similar to one or more embodiments of filters described in European patent EP0981311.

In this embodiment, the deodorizing filter 10 is shielded from the interior or main collection chamber of the ostomy appliance 1 by a gas permeable barrier-filter 11 having a first part 12 and a second part 15, where the outer surface of the first part 13 is secured to the inner surface 7 of the front-wall 6 and the outer surface 16 of the second part 15 is secured to the inner surface 3 of the back-wall 2 of the ostomy appliance. The first part 12 and the second part 15 of the barrier-filter 11 are positioned inside the ostomy appliance, so that the inner surface 14 of the first part 12 and the inner surface 17 of the second part 15 are substantially opposite each other, so that the inner surface area 14 of the first part 12 and the inner surface area 17 of the second part 15 are substantially overlapping.

The barrier-filter 11 can be made of two strips of Velcro, where the first part 12 is attached to the inner surface 7 of the front-wall 6 and the second part 15 attached to the inner surface 3 of the back-wall 2, using attachment means such as gluing, welding or other suitable means known in the art for attaching the barrier-filter. When in use, the gas will pass from the interior of the appliance 1, through the Velcro barrier 11 and through the deodorizing filter 10 via the opening 9 into the surrounding atmosphere or the open air.

The front-wall 6 and the back-wall 2 of the ostomy appliance 1 are permanently attached to each other on the inner surfaces 3,7 of each respective wall 2,6 along a connection area 16 where the walls 2,6 are attached to each other providing a permanent connection between the walls using means such as gluing, welding or other suitable means known in the art. The size, i.e. thickness, width or depth of the connection area 18, may differ from one embodiment to the other, where in this embodiment the area is at an offset from the distal edge towards the centre of the walls 2,6 providing an inner boundary 20 of the connection area 18.

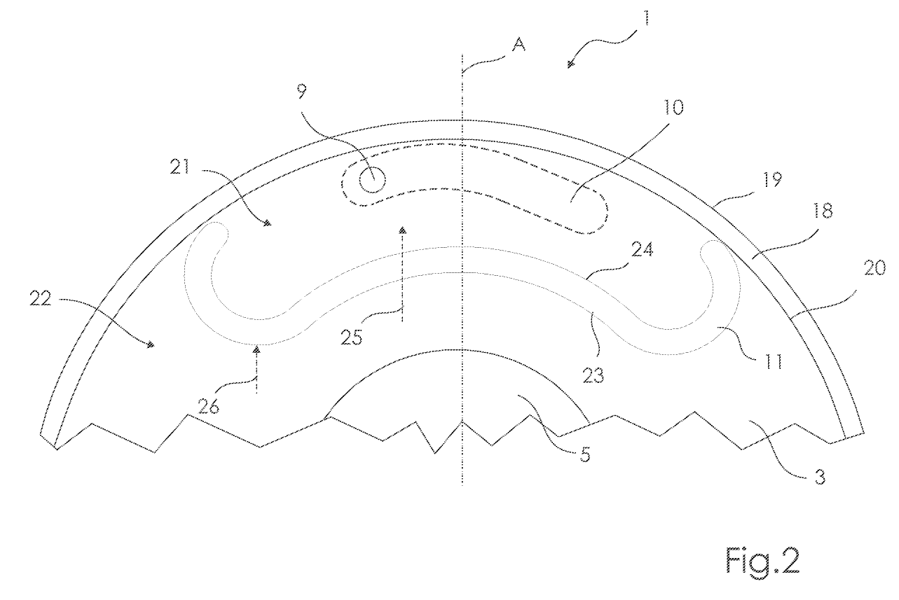

FIG. 2 shows a partial plan view of the ostomy appliance 1 according to the present invention having the barrier-filter 11 in a closed state. In the context of this figure and to promote understanding, the front wall 6 shown in FIG. 1 is not shown. In this embodiment, the front-wall (not shown) and the back-wall 3 are welded together, where the welded area radiates from the distal edge 19 towards the inner boundary 20 of the connection area 18.

The barrier-filter 11 is positioned so that it encloses an area containing the filter opening 9, separating the filter opening from the opening 5 in the back-wall 3. The barrier filter stretches from one inner boundary 20 of the weld 18 to another inner boundary 20 on the opposing side of the bag, across the axis A, defining an upper volume 21 which includes the filter opening 9 and a lower volume 22 which includes the opening 5 in the back-wall 3 and the waste-collecting part of the ostomy appliance 1. The barrier-filter 11 has a lower edge 23 and an upper edge 24 where the lower edge faces the lower volume 22 and the upper edge 24 faces the upper volume 21. In its closed state, the barrier-filter 11 allows gaseous material to communicate from the lower volume 22 penetrating the lower edge 23 of the barrier-filter 11 and exiting the barrier-filter via the upper edge 24 into the upper volume 21, as indicated using arrow 25. On the other hand, in its closed state, the barrier-filter 11 blocks any semi solid waste at the lower edge 23, as indicated with arrow 26, preventing the waste from entering the upper volume 21. In its open state, the barrier-filter 11 opens a direct communication pathway from the lower volume 22 to the upper volume 21 for all waste material collected in the ostomy appliance 1.

The barrier-filter 11 can be of a shape selected from the group of shapes consisting of: straight, curved, S-shaped, saw-toothed, bell shaped, E-shaped, a combination thereof or any suitable shape which may increase the dimensions of the lower edge 23 from one weld boundary 20 to the other. By increasing the dimension of the lower edge, the contact surface which has to be contaminated before the barrier-filter becomes clogged or blocked is increased as well. The endpoints of the barrier-filter 11 will not follow the contour, but connect the barrier to the inner boundary 20 of the weld or to a position near the inner boundary 20 of the weld so that semi-solid waste cannot pass between the inner boundary 20 and the barrier-filter 11.

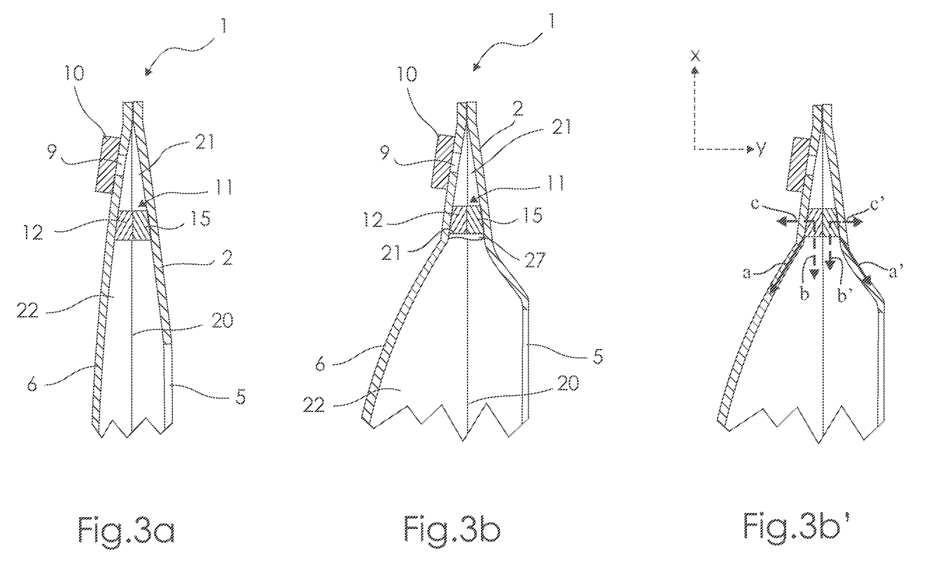

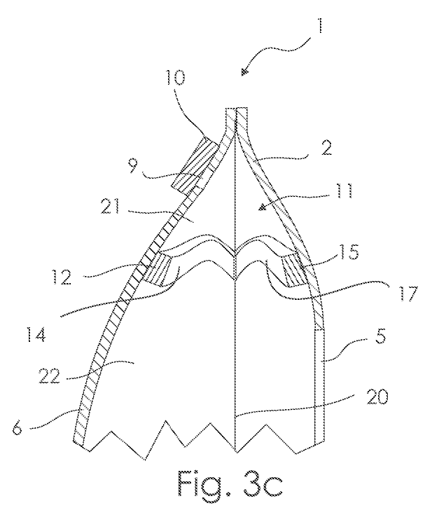

FIG. 3a shows a partial sectional view of an empty ostomy appliance 1 according to the present invention taken along line III-III of FIG. 1, where the barrier-filter, in a closed state 11, separates the upper volume 21 and the lower volume 22. If flatus gas or any other kind of gas would enter the ostomy appliance via the back-wall 2 opening 5, the gas would be able to penetrate the barrier-filter 11 and communicate from the lower volume 22 into the upper volume 21 and via the filter 10 into the open air.

FIG. 3b, on the other hand, shows a partial sectional view of a ballooned ostomy appliance according to the present invention, where the barrier filter 11 is clogged or blocked by semi-solid waste 27 collected on the lower edge 21 of the barrier filter 11. This means that the gaseous material, which has entered the ostomy appliance 1, cannot escape the appliance 1 via the filter 9 and the gas builds up in the lower volume 22. The gas causes the appliance 1 to balloon and the pressure inside the lower volume 22 increases as the volume of gas increases. As the ostomy appliance 1 balloons, the areas of the front-wall 6 and the back-wall 2 which are not attached to each other begin to separate away from each other: This separation causes increased tension in the walls 2,6 and when the separation of the walls 2,6 has reached a certain point, where the distance between the walls 2,6 is at its maximum without separating the barrier filter, the tensional forces present in the front-wall 6 and the back-wall 2 pull onto the first 12 and the second part 15 of the barrier filter 11, respectively.

FIG. 3b′ shows the orthogonal force vectors b,b′ and c,c′ that affect the barrier-filter via the tensional force vectors a,a′ which represents the tensional force in the front-wall 6 and the back-wall 2, respectively. The tensional force vectors a, a′ affect the barrier filter 11, by vector elements b,b′ and c,c′ in the directions defined by axis x and y, respectively. The vector elements b and b′ have little or no effect on the temporary connection between the first part 12 and the second part 15 of the barrier filter 11, as the vectors affect the first part and the second part in the same direction, −x. However, vector elements c and c′ show the forces which are pulling the temporary connection apart, as the vector elements c, c′ are affecting the first part 12 and the second part 15 in opposite directions y and −y. When the forces, shown as vector elements c and c′, are large enough to overcome the forces holding the temporary connection together, the temporary connection begins to separate and the first part 12 disconnects from the second part 15 and a communication pathway is opened between the lower volume 22 and the upper volume 21, as shown in FIG. 3c.

Based on the teachings of the present invention, it is obvious that by applying physical contact to the ostomy appliance this would result in the application of one or more of the tensional forces a, a′, b, b′, c and c′ on the front-wall 6 or the back-wall 2. This might be achieved by applying pressure to the external surface, as mentioned earlier, or by manually pulling the front-wall or the back-wall from each other, temporarily deforming the ostomy appliance and thereby achieving an amount of separation forces necessary for separating the barrier-filter.

FIG. 3c shows a partial section view along line III-III of FIG. 1 of the ostomy appliance 1 according to the present invention having the barrier filter 11 in an open state. In this open state, the inside surface 14 of the first part 12 and the inside surface 17 of the second part 15 are completely separated from each other at an area which is close to the sectional cut defined by the line III-III in FIG. 1. As the front-wall 6 and the back wall 2 are permanently welded to each other, as earlier described, the surface areas 14,17 of the first 12 and second part 15, respectively, may not be separated from each other in positions which are close to the inner boundary 20 of the weld.

The semi-solid waste 27 which blocked the barrier-filter 11, as shown in FIG. 3b, may come loose from the barrier filter when the barrier-filter 11 opens a passage. The waste 27 may disintegrate, break up, fall apart and come off in one piece or come off in at least two pieces of the barrier-filter 11 as a result of the mechanical or physical change on the barrier-filter 11. In a situation where the barrier-filter is very contaminated by waste, the waste may possibly not loosen at all. In those cases, it might be possible to remove the waste by administering physical stimulation using the fingers of the hand to the external surface of the ostomy appliance, pressing the waste away from the barrier filter. If the blockage cannot be removed by applying external stimulation, this might be considered as an indicator, indicating that the bag has to be replaced.

It is obvious from the above description and the teachings of the present invention that the transition of the barrier-filter's 11 from closed state to open state and vice versa occurs gradually using the internal pressure of the ostomy appliance. In a situation where a part of the lower edge 23 of the barrier-filter 11 begins to separate and portions of the inner surfaces 14,17 of the barrier filter are separated from each other and before the barrier-filter has opened a direct communication passage from the lower volume 22 to the upper volume 21, the separation may be enough to re-enable the gas permeability of the previously blocked barrier-filter 11.

It is also obvious from the above description and the teachings of the present invention that the barrier filter may be assumed to be in an open state at any level of separation of the inner surfaces 14,17 of the first part 12 and the second part 15, opening a direct communication pathway from the lower volume 22 and the upper volume 21. Likewise, the closed state of the barrier-filter 11 may be all degrees of temporary connection between the inner surfaces 14,17 which blocks the direct communication of semi-solid waste between the upper volume 21 and the lower volume 22, regardless of the actual gas permeability of the barrier-filter 11.

The present invention presents a barrier-filter 11 allowing more than one contamination to take place instead of being clogged permanently, as a conventional filter or barrier-filter would be. After the ostomy-appliance has been deflated, the barrier-filter will reach its closed state again protecting the deodorizing filter, until the pressure rises again inside the bag. This process can take place multiple times until the succeeding deodorizing filter or membrane is almost completely clogged with semi-solid waste.

### Example

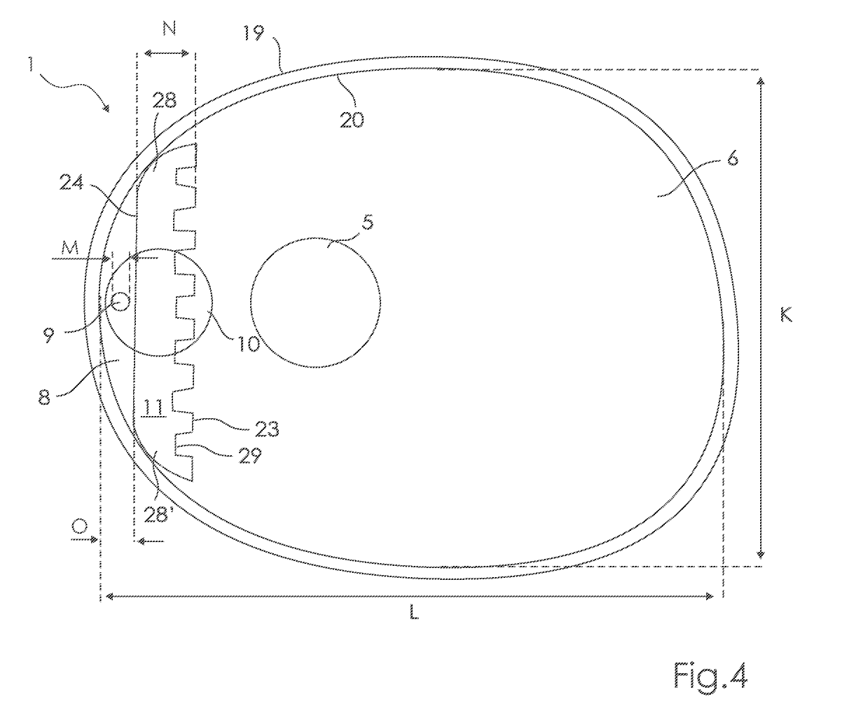

A functional example of an ostomy appliance 1 according to the present invention is shown in FIG. 4. FIG. 4 is a planar view of an ostomy appliance 1, where the parts of the ostomy appliance that are behind the front-wall 6 of the ostomy appliance 1, as seen in the viewing direction, are shown using dashed lines.

The front-wall 6 and the back-wall (not shown) are made of a substantially non-resilient flexible plastic material, approximately 75 μm thick, such as polyurethane, welded together, defining an inner boundary 18 of the weld. The width of the inner collection area of the ostomy appliance 18 measured from the inner boundary 20 of the weld and shown as the reference K is 146 mm. The length, shown with the reference L is 182 mm. The inner boundary 18 of the weld is substantially elliptical in shape. The diameter of the filter opening 9, shown using the reference M, is 5.5 mm and the deodorizing filter 10 is positioned on the outer surface 8 of the front wall 6, covering the filter opening 9 completely. The deodorising filter 10 is circular in shape having a diameter of approximately 30 mm. The deodorizing filter, in this specific example, is a Filtrodor® filter having a throughput of approximately 600 ml/min

The barrier-filter 11 of the ostomy appliance 1, is made out of an Aplix® 700 which is a hook and loop material made out of Polyolefin and having plastic hooks on the inner surface and corresponding loops on the opposing surface. The material has a width of 15 mm, shown using the reference N. The ends 28,28′ of the barrier-filter are positioned tightly close to the inner boundary 20 of the weld, so that no semi-solid material can communicate between the ends 28,28′ of the barrier-filter 11 and the inner boundary 18 of the weld. Furthermore, the lower edge 23 of the barrier-filter 11 is provided with seven indentations 29, which are cut 5 mm into the width of the barrier-filter 11 having a length of approximately 5 mm, increasing the length of the lower edge 23 by approximately 70 mm, from approximately 90 mm to 160 mm. The increase in length means that the barrier filter should tolerate more waste to contaminate the barrier-filter, before the filter becomes blocked or clogged. The maximum distance between the upper edge 24 of the barrier-filter to the inner boundary 18 of the weld, in an orthogonal direction from the upper edge 24, is approximately 10 mm shown using reference O, where the upper edge defines the upper volume of the ostomy appliance 1, as mentioned above.

This provides an ostomy appliance 1 according to the present invention, which separates the Velcro barrier-filter 11 using the tensional forces in the stretched walls at an inner pressure, approximately 1 kPa, opening a passage and allowing enclosed gasses to escape through the deodorising filter 10. As the pressure inside the ostomy appliance 1 decreases, the inner surfaces of the front wall 6 and the back wall (not shown) get closer to each other, and when the inner pressure is approximately 0 kPa the Velcro barrier-filter closes the opened passage.

1. An ostomy appliance of the kind comprising
a collecting bag for waste material
at least one opening through which waste material may enter the collecting bag
at least one filter allowing gas to exit the collecting bag, and
at least one pressure sensitive gas permeable barrier-filter positioned between the at least one opening and the at least one filter, where the barrier-filter in its closed state blocks the passage of semi-solid waste and in its open state maintains an open passage for semi solid waste,
where an increase in pressure inside the collecting bag exceeding a predefined first gas pressure limit triggers the transition of the barrier-filter from its closed state to its open state.

2. An ostomy appliance according to claim 1, wherein the barrier-filter is resealable.

3. An ostomy appliance according to claim 1, where the barrier-filter returns from its open state to its closed state when the pressure inside the collecting bag is decreased below a predefined second gas pressure limit.

4. An ostomy appliance according to claim 1, wherein the barrier-filter is a hook and loop fastener filter.

5. (canceled)

6. An ostomy appliance according to claim 1, wherein the shape of the barrier-filter is selected from the group of shapes consisting of: straight, curved, S-shaped, saw-toothed, bell shaped, E-shaped or a combination thereof.

7. A method of using an ostomy appliance according to claim 1, wherein the pressure inside the collecting bag is increased by applying pressure to the external surface of the collecting bag.

8. An ostomy appliance according to claim 1, wherein the collecting bag has at least a front-wall and a back-wall, each provided with a respective inner and outer surface.

9. An ostomy appliance according to claim 8, wherein the barrier-filter is secured to the inner surface of the front-wall and the back-wall of the ostomy appliance.

10. An ostomy appliance according to claim 9, wherein the barrier filter is laser welded, heat welded or adhesively bonded to the inner surface of the front-wall and the back-wall of the ostomy appliance.

11. An ostomy appliance according to claim 3, wherein the first pressure limit is in the range between 0.1 and 10 kPa.

12. An ostomy appliance according to claim 11, wherein the second gas pressure limit is in the range between 0 and 0.1 kPa.

13. An ostomy appliance according to claim 1, wherein the filter comprises at least one deodorizing gas filter.
